(19) United States
(12) Patent Application Publication
(10) Pub. No.: US 2019/0388480 A1
(43) Pub. Date: Dec. 26, 2019

(54) Method of Increasing Probiotic Viability in Food Products

(71) Applicant: Tropicana Products, Inc., Bradenton, FL (US)

(72) Inventors: Zeinab ALI, Crystal Lake, IL (US); Varadharajan Radhamani BASKER, Barrington, IL (US); Yumin CHEN, Gurnee, IL (US); Cordula FRANKRONE, Chicago, IL (US); Juan GONZALEZ, Barrington, IL (US); Prabhakar KASTURI, Barrington, IL (US); Yih Jennifer LEE, Port Barrington, IL (US); Tracy MUI, Buffalo Grove, IL (US); Elizabeth Gutierrez PRICE, Round Lake Park, IL (US); James D. SCHUMAN, Hoffman Estates, IL (US); Kelly VAN DYKE, Barrington, IL (US)

(21) Appl. No.: 16/015,668

(22) Filed: Jun. 22, 2018

(51) Int. Cl. A61K 35/741; A23L 33/135; A23L 2/02; A23L 2/52

(52) U.S. Cl. CPC A61K 35/741; A23L 33/135; A23V 2002/00; A23L 2/52; A23L 2/02

(57) ABSTRACT

A pre-conditioned probiotic composition and method of forming the pre-conditioned probiotic composition comprises introducing thawed probiotic cells into a pre-conditioning environment having sufficient nutrients and conditions to prepare the thawed probiotic cells for surviving and successfully thriving in a targeted food product. Thawed probiotic cells are then provided with an incubation period of at least 2 hours to produce inoculation pre-treated probiotic cells with improved shelf life in a food product.

### BACKGROUND OF THE INVENTION

### Technical Field

The present invention relates to food products comprising probiotic organisms; in particular, a method of producing robust probiotic cells to be incorporated into a variety of food products.

### Description of Related Art

In food products which are manufactured to incorporate living probiotic bacteria, there is often a substantial reduction of living probiotic cells (e.g. one log or more reduction) in certain food products by the end of the product's desired shelf life. One current solution to this reduction, when producing foods incorporating probiotics, involves overdosing the probiotics into the products to maximize the amount of remaining living probiotic bacteria over the shelf life of the food products. There remains a need for more robust probiotic cells or for a method for making probiotic cells robust in a wide variety of food products, so that overdosing of probiotics can be avoided to the greatest extent possible.

### SUMMARY OF THE INVENTION

Below is a simplified summary of this disclosure meant to provide a basic understanding of the method and product described herein. This is not an exhaustive overview and is not intended to identify key or critical elements or to delineate the scope of the description. Its sole purpose is to present some concepts in a simplified form as a prelude to the more detailed description below.

A method of producing robust probiotic cells comprises the steps of introducing thawed probiotic cells into a pre-conditioning environment, the pre-conditioning environment comprising sufficient nutrients to prepare the thawed probiotic cells for incorporation into a targeted food product; and providing the thawed probiotic cells with an incubation period of at least 2 hours, thereby producing inoculation pre-treated probiotic cells. In some embodiments, the incubation period comprises up to 1 whole probiotic cell division cycle. In some embodiments, prior to the introducing step, the method comprises a step of thawing frozen probiotic cells. In some embodiments, the method further comprises, following the incubation period, the step of adding the pre-treated probiotic cells to the targeted food product to produce a food product comprising robust probiotic supplements. Optionally, the treating and pre-conditioning steps take place simultaneous with the production of the target food product (i.e., food or beverage product) to which the probiotic bacteria is added. Inoculated probiotic cells may be incorporated with any number of fruits, vegetable, and/or dairy products. In some embodiments, the food product comprises a low pH matrix, which may comprise a pH of less than about 4.5.

Other aspects, embodiments and features of the invention will become apparent in the following written detailed description and accompanying drawings.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 depicts a flowchart for one embodiment of the method described herein.

FIG. 2 depicts a schematic explanation of the toxicity of low pH food product environments to probiotic cells and the detoxification mechanism of cellular processes of the method described herein.

FIG. 3 depicts a bar chart comparison of three samples comparing viability of probiotic cells in which probiotics are introduced to a beverage by direct inoculation without pre-conditioning, pre-conditioning for 2 hours at 4° C., and pre-conditioning for 4 hours at 4° C.

### DETAILED DESCRIPTION OF THE INVENTION

Currently available probiotics are produced under conditions ideal for subsequent storage by freezing or freeze drying until use is desired. Such conditions are extremely different from the conditions of some food product matrices that probiotic bacteria are incorporated into. By way of example, freshly thawed probiotic bacteria are not prepared for shelf life survival in the acidic conditions found in fruit juices such as orange juice, which comprises a pH level of about 4, an aerobic environment, compounds that can be harmful to probiotic viability, and nutrient levels which may not be rich in the amino acids or proteins to which the probiotics are adapted for survival. For example, phytochemicals (e.g. polyphenols) can negatively affect probiotic viability. The probiotic bacteria cells thawed from frozen state lack the structure and enzymatic system to handle the low pH environment of fruit-based beverages, including proton pumps on the cell membrane (F₀F₁-ATPase) and the cellular buffering system. Consequently, the shelf life of the probiotic cells in the food product significantly decreases.

Under such vastly different beverage product conditions, there is usually greater than about 95% reduction of living probiotic bacteria cells over a 72 day period following incorporation of living probiotic bacterial cells into acidic beverages, such as fruit-based beverages. The root cause of probiotics low survivability in fruit-based beverages is that probiotic bacterial cells are produced under conditions that are designed to maximize their yield and viability after storage; that is, the probiotic cells are designed to maximize recovery from the damage of the freeze-thaw cycle. However, these conditions are not designed to allow the probiotic cells to adapt for survival in the product matrix of and under the production conditions of certain food products, including fruit-based beverages comprising a low pH. There is no time and no nutrient support for the probiotic cells to adapt to their destined/final food product matrix. Fruit-based beverages comprising low amounts of amino acids or proteins, for example, cannot provide sufficient nutrients for probiotic bacteria to produce the much-needed proton pumps, cellular buffering system, and enzymes for detoxifying oxygen that allow the cells to survive in a harsh, low pH environment.

There are four phases of bacterial growth cycle: lag phase, log phase, stationary phase, and death phase. During the lag phase, bacterial cells replicate various proteins and synthesize RNA in preparation for the log phase, during which the size of the bacterial colony increases dramatically. Following the log phase, the stationary phase involves a slow-down in the growth of the cells and the colony. As resources are used, the rate of cell death begins to match the rate of cell division. As conditions decline due to limited resources, waste production or other environmental changes, the colony enters the final phase of death.

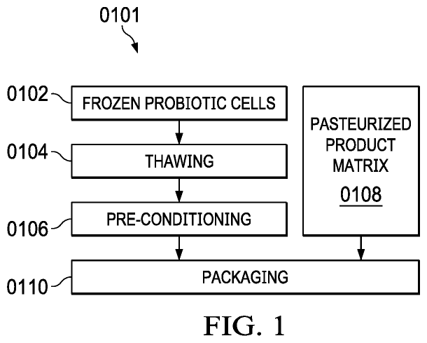

By focusing on the conditions and physiology of the lag phase, it is proposed to treat the probiotics to allow for the adaptation to certain growth conditions of the food matrix to which the probiotic bacteria are ultimately incorporated. By providing components to the bacteria that will allow for stabilization of its medium to a proper balance, improved interactions with certain food matrices will result in improved shelf life of the probiotics. FIG. 1 is a flow chart illustrating one embodiment of a method useful for increasing the viability of probiotics in a pasteurized product matrix 0108.

By incorporating nutrient processing aids such as carbohydrates and amino acids, and setting the proper conditions for probiotic cells to build cellular enzyme systems, treated probiotic cells are set for survival at a low pH, in one embodiment. The method for forming stable or robust probiotic cells comprises providing the frozen probiotic cells 0102. The frozen probiotic cells 0102 can be stored in a freezer at −80° C. In some embodiments, the method comprises thawing 0104 the frozen probiotic cells 0104. For example, the frozen probiotic cells 0102 can be placed in a storage environment in a temperature of 0-39° C. In some embodiments, the method comprises introducing the thawed probiotic cells into a pre-conditioning environment 0106, the pre-conditioning environment comprising sufficient nutrients to prepare the thawed probiotic cells for incorporation into a pasteurized food product; and pre-conditioning the thawed probiotic cells with an incubation period of at least 2 hours, thereby producing inoculation pre-treated probiotic cells. In some embodiments, the method can comprise of thawing frozen probiotic cells prior to introducing the thawed probiotic cells into the pre-conditioning environment. In some embodiments, the method can comprise of thawing frozen probiotic cells at the same time as introducing the thawed probiotic cells into the pre-conditioning environment. For example, the combined step of thawing and introducing can take about 2 to 24 hours. The conditions of the pre-conditioning environment may provide for recovery of the probiotic cells from freezing-thawing damage of manufactured frozen probiotic cells. In some embodiments, the pre-conditioned probiotic cells are introduced into a pasteurized product matrix 0108 to provide a food product comprising probiotic cells. As used herein, a “pasteurized product matrix” may be in semisolid, or liquid form (e.g., a snack food or a beverage). As used herein, a “food product” comprises the pasteurized product matrix and pre-conditioned probiotic cells. In some embodiments, the method comprises packaging 0110 the food product in an aseptic container.

To obtain robust probiotic cells, in some embodiments, sufficient nutrients include one or more of carbohydrates and amino acids. In some embodiments, the nutrients comprise carbohydrates such as, for example, lactose, maltose, fructose, glucose, sucrose and a combination thereof. In some embodiments, the nutrients comprise other simple sugars or oligosaccharides naturally found in juices. For example, juices, concentrates, purees, nectars, and other preparations may be derived from any one or a combination of fruits such as apple, orange, pear, mango, pomegranate, watermelon, pineapple, peach, grape, kiwi, coconut, and lemon. In some embodiments, the nutrients comprise carbohydrates such as, for example, juices or purees derived from vegetables such as wheat grass, cucumber, lettuce, pumpkin, and winter melon. In some embodiments, the nutrients comprise one or more amino acids. In some embodiments, the amino acids comprise homocysteine. In some embodiments, amino acids comprise cystathionine. In some embodiments, amino acids comprise cysteine. In some embodiments, amino acids comprise homocysteine, cystathionine, cysteine, or any combination or subcombination thereof. Other suitable amino acids include without limitation alanine, isoleucine, leucine, valine, phenylalanine, tryptophan, tyrosine, asparagine, cysteine, glutamine, methionine, serine, threonine, arginine, histidine, lysine, aspartic acid, glutamic acid, glycine, proline or any combination or subcombination thereof. Not being bound to any particular theory, it is believed that the amino acids allow for the probiotic cells to produce proton pumps and develop cellular buffers to cope with low pH conditions of a food product to which the probiotic will be added to produce a food product with robust probiotic supplements.

In addition to carbohydrates and amino acids, organic acids may also be present in suitable pre-conditioning environments. A few examples of organic acids may include, but are not limited to citric acid, malic acid, tartaric acid, succinic acid, formic acid and lactic acid, or combinations or subcombinations thereof.

In addition to sufficient balanced nutrients for successful thriving in a desired food product matrix environment, the method comprises providing the thawed probiotic cells with an incubation period of time of between about 2 hours and up to the time it takes the probiotic cells to complete one cell divisional cycle. In some embodiments, the cell divisional cycle may comprise up to about 24 hours. In some embodiments, the cell divisional cycle may be between about 6 to 16 hours. The cell divisional cycle time is dependent on a number of different factors, including, for example, the environment of the probiotic cells, the quality of pre-conditioning fluid, and the type of probiotic cells. The incubation period should allow and induce probiotic cells to produce proton pumps and develop the cellular buffers needed to cope with a food product, more specifically to cope with the environment of the targeted or desired food product to which the pre-treated probiotic cells will be added. Not wishing to be bound by any particular theory, it is believed that an incubation period that extends beyond the cell divisional cycle may result in a product with sensory attributes and nutritional values that deviate from the desired product specification. For example, the cell division process may include further metabolizing of nutrients in the environment, resulting in changes in the nutritional value and sensory profile of the product.

Treatment within the pre-conditioning environment may also comprise suitable temperatures to allow the probiotic cells to adapt to a desired food product matrix. In some embodiments, the pre-conditioning environment comprises a temperature of between about 0° C. and 39° C. In some embodiments, the pre-conditioning environment comprises a temperature of about 4 to 10° C. In other embodiments, a temperature or temperatures may be selected to balance the energy cost of warming the cells and optimize the time it takes to thaw. In some embodiments, the time it takes to thaw is about 1 to 16 hours. In some embodiments the time it takes to thaw is from 8 to 16 hours. In some embodiments, the time it takes to thaw is about 4 to 8 hours. The time it takes to thaw can vary depending on the temperature used to thaw the probiotics.

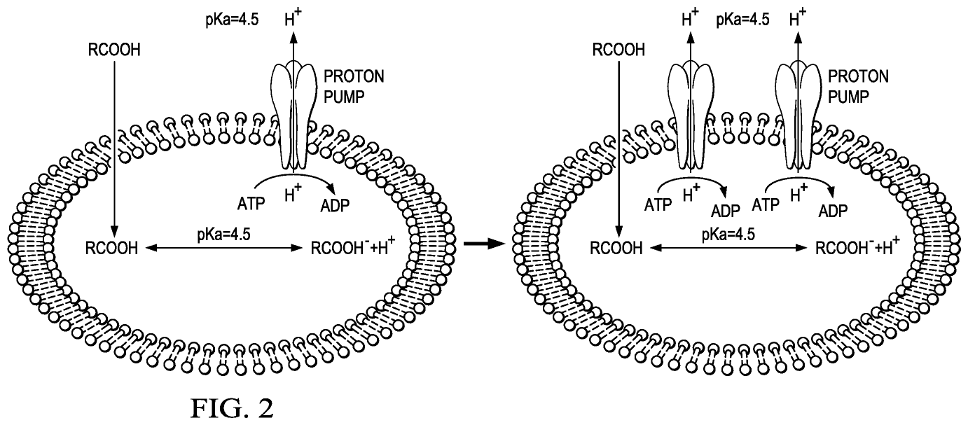

In some embodiments, the process 0101 shown in FIG. 1 may be performed in parallel with blending and pasteurization process of fruit beverages or other food products. FIG. 2 depicts a schematic explanation of the toxicity of the low pH environment of a fruit beverage to probiotic cells, the detoxification mechanism of proton pump, and the key cellular process underlying the treating of the probiotic cells described herein. Outside a cell, a sample fruit beverage comprises a pH of 4. The low outside pH favors a significant portion of organic acid in the neutral form. The neutral form of this organic acid diffuses into the cell. Inside the cell, a high internal pH (pH 6.5) favors a significant portion of organic acid to dissociate to form H^{|} and the organic acid anion. H^{|} is toxic to the cell. By providing energy in the form of carbohydrates, building blocks in the form of amino acids, suitable temperatures, and time for cells to synthesize more proton pumps (right side of FIG. 2), the cell can better adapt to low pH and is then better equipped to cope with excess protons by pumping them out.

In some embodiments, the food product comprises fruit juice beverages, carbonated beverages, fermented beverages, a fortified water, a fruit smoothie, a vegetable juice, a dairy smoothie, a dairy beverage, an energy drink, a sachet, a shot, or a yogurt product. A “shot” refers to a volume of about 4 ounces or less of a liquid product formulation. In some embodiments, the food product is a “snack food product,” which refers to a ready-to-eat food product that requires no further cooking. In some embodiments, a produced snack food requires refrigeration. It should be understood that food products in accordance with this disclosure can have any of numerous different specific formulations or components.

In some embodiments, the food product is a beverage. A beverage includes, for example, juice beverages (e.g., beverages comprising one or more fruit juices and/or one or more vegetable juices), hydration beverages such as those with added electrolytes, frozen or chilled beverages, caffeinated beverages, carbonated beverages, non-carbonated beverages, zero to low calorie drinks (for example, 0-150 kcals and up to 10 grams sugar/12 oz), such as diet or other reduced calorie beverages, and syrups or concentrates. Non-limiting examples of suitable dairy-containing beverages include milk (e.g., 2% milk) and other beverages containing milk (e.g., coffee drinks containing milk). Other suitable dairy-containing beverages include any of those known in the art. In some embodiments, the food product is a beverage that may require refrigeration. In some embodiments, the food product is beverage stable and safe to store at ambient conditions not requiring refrigeration. In some embodiments, the food product comprises a low pH of less than about 4.5. Suitable beverages may comprise sweetener, water, dairy, caffeine, carbonation, fruit juice, vegetable juice, food grade acid, or a mixture thereof. Beverage products disclosed herein include ready-to-drink liquid formulations (i.e., ready-to-drink beverages), beverage concentrates, beverage pods, and the like. The term “ready-to-drink” refers to a beverage formulated to be ingested as-is. Thus, in some embodiments, the ready-to-drink beverage requires no dilution or additions prior to ingestion by a consumer. It should be understood that beverages and beverage concentrates in accordance with this disclosure can have any of numerous different specific formulations or constitutions.

### EXAMPLES

A 750 g bag of probiotic was stored in a freezer at −80° C. until ready for use. The sample was then taken from the freezer and placed in a storage environment with a temperature of 4° C. for 16 hours to thaw. The thawed probiotic was mixed with pasteurized single strength apple juice that was chilled to 4° C. prior to mixing. The probiotic apple juice mixture had about 9.7×10¹⁰ CFU/mL of live probiotic cells (approx. 1 to 1 ratio for concentrated stock to start with). After the mixture was pre-conditioned at 4° C. for 2 hours in one example, or 4 hours in another example, the probiotic cells were inoculated into 32 oz (946 mL) of strawberry banana juice base to reach at approximately 10⁸ CFU/mL live probiotic cells. As a control, another sample of the strawberry banana juice base was mixed with the same amount of non-preconditioned probiotic cells. For the control sample, an amount of non-preconditioned probiotic cells were added to the strawberry banana juice base at the same level as the inoculated samples containing the pre-conditioned mixture (e.g. approximately 10⁸ CFU/mL live probiotic cells). After inoculation, the two examples and one control sample of probiotic strawberry banana juice were stored at 4° C. The amount of live probiotic cells in the juice after inoculation was measured immediately upon inoculation, then after 2 days, 14 days, 30 days, 42 days, 65 days and 72 days of storage at 4° C.

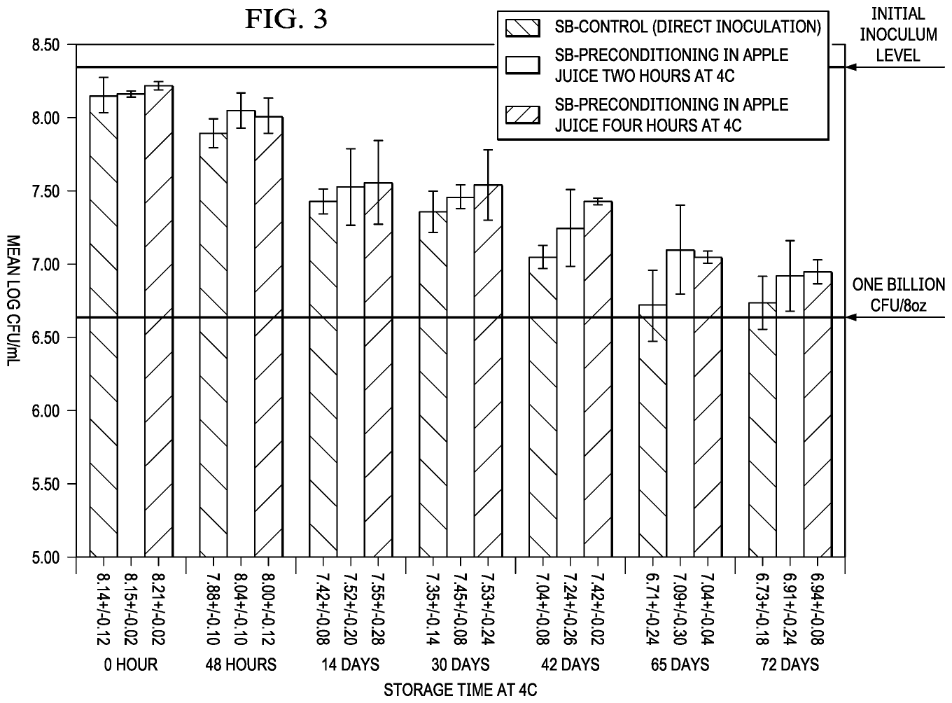

FIG. 3 shows the mean log CFU/mL measurements of live probiotics at each time interval. In all three samples, the attrition rate of the live probiotic cells over the 65 day period was gradual. However, the rate of attrition seen in the non-preconditioned control sample was greater than the pre-conditioned samples. For example, after 65 days, the remaining colony forming units as a percentage of the initial CFU in the pre-conditioned samples were double the percentage of the remaining CFU in the non-preconditioned control sample. The pre-conditioned sample results indicated that it is possible to reduce the initial amount of probiotic cells introduced into the base in order to achieve a product that satisfies the claim of providing 1 billion CFU of probiotic cells in an 8 oz product (236 mL) by the shelf life date.

### Additional Embodiments

The following descriptive embodiments are offered as further support of the disclosed invention:

In a first embodiment, novel aspects described in the present disclosure are directed to a method of forming stable probiotic cells comprising: introducing thawed probiotic cells into a pre-conditioning environment, the pre-conditioning environment comprising sufficient nutrients to prepare the thawed probiotic cells for incorporation into a food product; and pre-conditioning the thawed probiotic cells in the pre-conditioning environment for at least about 2 hours, thereby producing pre-conditioned probiotic cells.

In another aspect of the first embodiment, the method of forming stable probiotic cells comprising: introducing thawed probiotic cells into a pre-conditioning environment, the pre-conditioning environment comprising sufficient nutrients to prepare the thawed probiotic cells for incorporation into a food product; pre-conditioning the thawed probiotic cells in the pre-conditioning environment for at least about 2 hours, thereby producing pre-conditioned probiotic cells; and further comprises one or more limitations selected from the following:

wherein the pre-conditioning environment comprises apple juice;

wherein the pre-conditioning environment comprises a fluid and wherein the fluid and probiotic cells are blended to achieve a predetermined probiotic concentration;

wherein the predetermined probiotic concentration is between about 1.0×10¹⁰ CFU/mL and 1.0×10¹¹ CFU/mL;

wherein the pre-conditioning environment comprises a temperature of between about 0° C. and about 39° C.;

wherein the pre-conditioning environment comprises a temperature of between about 0° C. and about 10° C.;

wherein the incubation period is less than one complete cell division cycle;

wherein the at least one complete cell division cycle is less than 24 hours;

prior to the introducing step, thawing frozen probiotic cells;

thawing frozen probiotic cells at a temperature of about 0-39° C.;

thawing frozen probiotic cells for at least 8 hours;

following the providing step, adding the pre-treated probiotic cells to a pasteurized product matrix to produce a food product comprising stable probiotic cells;

wherein the nutrients comprise carbohydrates;

wherein the nutrients comprise amino acids;

wherein the amino acids comprise homocysteine, cystathionine, cysteine, or combinations thereof.

In a second embodiment, novel aspects of the present disclosure are directed to a pre-conditioned probiotic blend for incorporation into a pasteurized product matrix to provide a food product comprising: a predetermined amount of thawed probiotic cells; a pre-conditioning fluid, wherein the pre-conditioning fluid comprises nutrients; and a probiotic concentration between about 1.0×10¹⁰ and about 1.0×10¹¹ CFU/mL.

In another aspect of the second embodiment, novel aspects of the present disclosure are directed to a pre-conditioned probiotic blend for incorporation into a pasteurized product matrix to provide a food product comprising: a predetermined amount of thawed probiotic cells; a pre-conditioning fluid, wherein the pre-conditioning fluid comprises nutrients; and a probiotic concentration between about 1.0×10¹⁰ and about 1.0×10¹¹ CFU/mL, the pre-conditioned probiotic blend for incorporation into a pasteurized product matrix to provide a food product further comprising one or more limitations selected from the following:

wherein the pasteurized product matrix comprises a pH of less than about 7.0;

wherein the pasteurized product matrix comprises a pH between about 4.0 and about 5.0;

wherein the pasteurized product matrix comprises a first pH and the pre-conditioned probiotic blend comprises a second pH, wherein the first pH differs from the second pH by 1.0 pH or less;

wherein the food product is a snack food product;

wherein the food product is a beverage;

wherein the beverage comprises dairy;

wherein the beverage comprises fruit;

wherein the beverage comprises vegetable;

wherein the beverage comprises caffeine;

wherein the beverage comprises carbonation;

wherein the beverage provides at least 1 billion CFU probiotic cells per 8 oz (236 mL) serving;

wherein the beverage provides between about 1.6×10⁸ CFU/mL and about 2.6×10⁸ CFU/mL of live probiotics;

wherein the nutrients comprise carbohydrates;

wherein the nutrients comprise amino acids;

wherein the nutrients comprise organic acids;

wherein the pre-conditioning fluid is derived from a vegetable; and wherein the pre-conditioning fluid is juice derived from a fruit;

In another aspect of the invention, a food product (for example, a beverage) comprises a preconditioned live probiotic blend comprising any combination or subcombination of features described above.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition is expressly set forth in the specification in a definitional manner that directly and unequivocally provides the special definition for the term or phrase.

The terms “including,” “comprising,” “having,” and variations thereof mean “including but not limited to,” unless expressly specified otherwise. When used in the appended claims, in original and amended form, the term “comprising” is intended to be inclusive or open-ended and does not exclude any additional, unrecited element, method, step or material. The term “consisting of” excludes any element, step or material other than those specified in the claim. As used herein, “up to” includes zero, meaning no amount (i.e., 0%) is added in some embodiments.

Unless specifically set forth herein, the terms “a”, “an”, and “the” are not limited to one of such elements, but instead mean “at least one,” unless otherwise specified. The term “about” as used herein refers to the precise values as indicated as well as to values that are within statistical variations or measuring inaccuracies.

The methods disclosed herein may be suitably practiced in the absence of any element, limitation, or step that is not specifically disclosed herein. Similarly, specific product embodiments described herein may be obtained in the absence of any component not specifically described herein. Thus, the crisps described herein may consist of those listed components as described above.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. For example, the range 1 to 10 also incorporates reference to all rational numbers within that range (i.e., 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

While this invention has been particularly shown and described with reference to several embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method of forming stable probiotic cells comprising:
introducing thawed probiotic cells into a pre-conditioning environment, the pre-conditioning environment comprising sufficient nutrients to prepare the thawed probiotic cells for incorporation into a food product; and
pre-conditioning the thawed probiotic cells in the pre-conditioning environment for at least about 2 hours, thereby producing pre-conditioned probiotic cells.

2. The method of claim 1 wherein the pre-conditioning environment comprises apple juice.

3. The method of claim 1, wherein the pre-conditioning environment comprises a fluid and wherein the fluid and probiotic cells are blended to achieve a predetermined probiotic concentration.

4. The method of claim 3, wherein the predetermined probiotic concentration is between about 1.0×10¹⁰ CFU/mL and 1.0×10¹¹ CFU/mL.

5. The method of claim 1 wherein the pre-conditioning environment comprises a temperature of between about 0° C. and about 39° C.

6. The method of claim 1 wherein the pre-conditioning environment comprises a temperature of between about 0° C. and about 10° C.

7. The method of claim 1, wherein the incubation period is less than one complete cell division cycle.

8. The method of claim 7, wherein the at least one complete cell division cycle is less than 24 hours.

9. The method of claim 1 comprising, prior to the introducing step, thawing frozen probiotic cells.

10. The method of claim 9 comprising, thawing frozen probiotic cells at a temperature of about 0-39° C.

11. The method of claim 9 comprising, thawing frozen probiotic cells for at least 8 hours.

12. The method of claim 1 comprising, following the providing step, adding the pre-treated probiotic cells to a pasteurized product matrix to produce a food product comprising stable probiotic cells.

13. The method of claim 1 wherein the nutrients comprise carbohydrates.

14. The method of claim 1 wherein the nutrients comprise amino acids.

15. The method of claim 14 wherein the amino acids comprise homocysteine, cystathionine, cysteine, or combinations thereof.

16. A pre-conditioned probiotic blend for incorporation into a pasteurized product matrix to provide a food product comprising:
a predetermined amount of thawed probiotic cells;
a pre-conditioning fluid, wherein the pre-conditioning fluid comprises nutrients; and
a probiotic concentration between about 1.0×10¹⁰ and about 1.0×10¹¹ CFU/mL.

17. The probiotic blend of claim 16 wherein the pasteurized product matrix comprises a pH of less than about 7.0.

18. The probiotic blend of claim 16 wherein the pasteurized product matrix comprises a first pH and the pre-conditioned probiotic blend comprises a second pH, wherein the first pH differs from the second pH by 1.0 pH or less.

19. The probiotic blend of claim 16 wherein the food product is a beverage.

20. The probiotic blend of claim 16, wherein the food product provides at least 1 billion CFU probiotic cells per 8 oz (236 mL) serving.
